(19) United States
(12) Patent Application Publication
(10) Pub. No.: US 2007/0190188 A1
(43) Pub. Date: Aug. 16, 2007

(54) Controlling Toxicity of Aminoquinoline Compounds

(75) Inventors: Larry Walker, Oxford, MS (US); Babu Tekwani, Oxford, MS (US); Lalit M. Tripathi, Lucknow (IN)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(73) Assignee: The University of Mississippi, University, MS (US)

(21) Appl. No.: 11/610,343

(22) Filed: Dec. 13, 2006

Related U.S. Application Data
(60) Provisional application No. 60/750,123, filed on Dec. 13, 2005.

(51) Int. Cl. A61K 31/47; A61K 31/496; A61K 36/752

(52) U.S. Cl. 424/736; 514/311; 424/777; 514/28; 514/254.07; 514/253.08; 514/312

(57) ABSTRACT

A method of controlling toxicity to a user caused by administration to the user of an aminoquinoline compound comprising administering to the user a toxicity controlling amount of at least one inhibitor of a cytochrome P450 (CYP) enzyme.

### CROSS REFERENCE TO RELATED APPLICATION

This application claims priority from U.S. provisional patent application Ser. No. 60/750,123, titled Method of Controlling Toxicity of Aminoquinoline Compounds, filed Dec. 13, 2005, and the complete content of that application is incorporated by reference.

### FIELD OF THE INVENTION

The present invention relates to the field of aminoquinoline compounds and toxicity problems associated with their use as medication.

### BACKGROUND OF THE INVENTION

The diseases caused by infections with parasitic protozoa are responsible for considerable mortality and morbidity affecting more than 500 million people in the world. The infections with parasitic protozoa namely Plasmodium spp., Leishmania spp. and Trypanosoma spp. are more prevalent in tropical and subtropical countries causing heavy loss of lives and reduced working abilities. Despite this heavy burden on humanity only a few antiparasitic drugs have been developed during last several years (Linares, Ravaschino and Rodriguez 2006). Aminoquinoline compounds, such as “8-Aminoquinolines”, are an important class of anti-infective drugs with promising utility in treatment of malaria and other emerging infectious diseases (Tekwani & Walker, 2006). The primary safety concerns with these drugs are methemoglobinemia and hemolytic events, particularly in populations with glucose 6-phosphate dehydrogenase deficiencies (Coleman and Coleman, 1996). Recent studies have shown potential for development of stereoselective analogs with better efficacy and reduced toxicity. NPC1161, a dichlorophenoxy derivative of primaquine, has been identified as a promising candidate with enantioselective toxicity and efficacy profiles (Tekwani and Walker, 2006) NPC1161 B, the more efficacious (−) enantiomer, shows considerably reduced toxicity than NPC1161A, the more toxic and less efficacious (+) enantiomer.

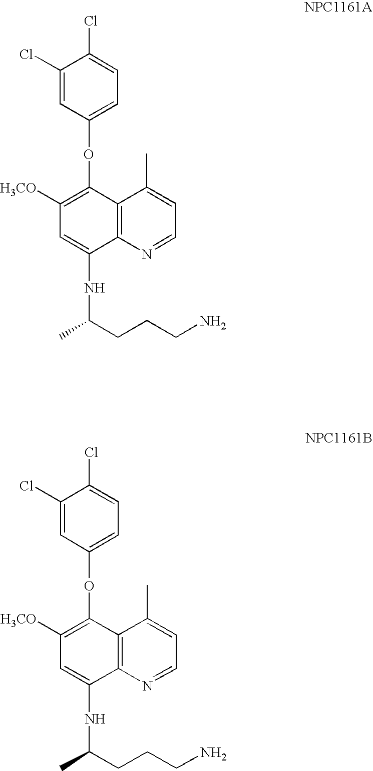

Biotransformation mechanisms, which appear to be central to anti-infective efficacies and hematological toxicities of 8-aminoquinolines, are still not well understood (Brueckner, Ohart, Baird et al. 2001). Reactive and unstable properties of potential methemoglobinemic metabolites (MtHbM) have hampered the studies on biotransformation mechanisms involved in toxicity of 8-aminoquinolines. Understanding of these mechanisms may help in developing the therapeutic strategies for control of this important toxic manifestation, which occurs during treatments with 8-aminoquinolines.

### DESCRIPTION OF THE INVENTION

One aspect of the present invention is a method of reducing toxicity of aminoquinoline compounds.

Another aspect of the present invention is a method for controlling toxicity of 8-aminoquinoline compounds that are primarily metabolized by cytochrome P450 3A4 (CYP3A4), comprised of the administration of inhibitors of CYP 3A4 (Zhou et al., 2005; Zhou et al., 2004). Examples of these inhibitors include macrolide antibiotics and other clinically used inhibitors, including but not limited to: cimetidine, ketoconazole, fluoroquinolone antibacterials, and HIV-protease inhibitors.

In another aspect of the present invention, the inhibitors are mechanism-based inhibitors.

In another aspect of the present invention, the toxicity controlled is extended to the methemoglobin and hemolytic toxicity of other 8-aminoquinolines which are metabolized by other cytochrome P450s (CYPs), and is comprised of administration of available inhibitors of other CYP enzymes, including but not limited to: CYP1A2, CYP2D6, CYP3A4, CYP2B6, and CYP2E1.

In one aspect of the present invention, an in vitro assay is developed for evaluation of biotransformation reactions leading to generation of methemoglobinemic metabolites of 8-aminoquinolines. This method uses simultaneous incubation of a test compound with the hepatic microsomes or Baculosomes® (microsomes prepared from insect cells infected with recombinant baculovirus containing a cDNA insert for a specific human P450 isozyme and NADPH-P450 reductase) and human erythrocytes followed by determination of methemoglobin formation. This allows the unstable metabolites generated in situ to react with the human erythrocytes. As shown in FIGS. 1-3, the 8-aminoquinolines generate methemoglobin in the presence of mouse or human liver microsomes, but not in the absence of liver microsomes. The response is related to the concentration of the 8-aminoquinoline (FIG. 3). The method is useful to study the biotransformation mechanisms involved in methemoglobin toxicity of 8-aminoquinolines and other agents known to cause hematotoxicity.

A comparative evaluation between primaquine, an 8-aminoquinoline in clinical use, and also the two enantiomers of an investigational 8-aminoquinoline NPC 1161 indicates that the metabolic mechanisms involved in clearance of these drugs and those responsible for their hematological toxicity are different. The microsomal cytochrome P-450 linked mixed function oxidase system plays an important role in metabolism of 8-aminoquinolines to potential metabolites responsible for causing methemoglobinemia. NPC 1161B shows significantly reduced methemoglobin toxicity than primaquine and NPC 1161A (FIGS. 1 and 2; Table 1). The enantiomers of NPC1161 are differentially recognized by human cytochrome P-450 drug metabolism systems. NPC1161B (the developmental candidate) is primarily metabolized by CYP 3A4, while other CYPs also contribute to MetHb toxicity of PQ and NPC1161A (FIG. 4, Table 2). CYP 3A4 generates almost similar methemoglobin toxicity with Primaquine, NPC1161A and NPC1161B (FIG. 5). The methemoglobin toxicity of NPC1161B could be almost completely abolished with macrolide antibiotics, for example, troleandomycin and erythromycin, the mechanism based inhibitors of CYP 3A4 (FIG. 7 and 8). Significant control of methemoglobin toxicity due to primaquine and NPC 1161A could also be achieved with the macrolide antibiotics (Tables 3-6) and also with other clinically used inhibitors of CYP 3A4 such as, for example, ketoconazole and cimetidine (Table 6, FIG. 9).

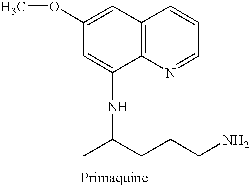

Accordingly, one aspect of the present invention is a novel metabolic mechanism based approach, which comprises the use of mechanism-based inhibitors of CYP 3A4 including macrolide antibiotics and other clinically used inhibitors, to control hematological toxicity of the 8-aminoquinolines.

The agents of the present invention can be delivered in several manners. For example, the agents or compositions of the present invention may be, or be part of a pharmaceutical composition, which will also comprise carriers or excipients that facilitate the processing of the present invention. Additionally, the agents of the preset invention can be co administered with the aminoquinoline preparation. In one embodiment, the agents can be administered in an amount effective for inhibition of CYP-mediated drug metabolism.

Determination of the effective amounts is well within the capability of those skilled in the art, especially in light of the detailed disclosure provided herein. That is, the amount of composition administered will be dependent upon the condition being treated, the subject being treated, on the subject's weight, the severity of the affliction, the manner of administration and the judgment of the individual's physician. As an example, dose schedules can be adjusted in relation to the dosing of the 8-aminoquinolines to prevent the methemoglobin response. The effective amount of the cytochrome P450 (CYP) inhibitor comprises an amount effective to control the toxicity of the aminoquinoline compound. The effective amount of the aminoquinoline is that amount effective to provide an anti-infective result, for example, an amount effective to treat malaria or other emerging infectious diseases.

In one embodiment of the present invention, the agents can be administered orally, using currently available dosage forms such as, for example, macrolide antibiotics, cimetidine, ketoconazole, HIV-protease inhibitors, and other drugs already approved for other indications.

As an additional example, one embodiment of the present invention can include coadministration orally of erythromycin stearate in a dosage up to 4 grams per day and primaquine in a dosage up to 40 mg per day throughout the treatment period to reduce the toxicity of primaquine.

### BRIEF DESCRIPTION OF THE FIGURES

FIG. 1. Formation of methemoglobinemic metabolites from 8-aminoquinolines by incubation with pooled human liver microsomes in vitro. Each point represents mean value of four observations.

FIG. 2. Formation of methemoglobinemic metabolites from 8-aminoquinolines by incubation with mouse liver microsomes in vitro. Each point represents mean value of four observations. FIGS. 3A and 3B Dose response study with primaquine and NPC1161B for generation of methemoglobinemic metabolites in vitro in presence of pooled human liver microsomes. Each point represents mean±S.D. of at least four observations.

FIG. 4. Involvement of different human CYP isoforms in methemoglobin toxicity of 8-aminoquinolines. The values on y axis show % methemoglobin formed and the values are mean±S.D. of at least four observations.

FIG. 5. Formation of methemoglobinemic metabolites from 8-aminoquinolines by incubation with 3A4 baculosomes® (microsomes prepared from insect cells infected with recombinant baculovirus containing a cDNA insert for human 3A4 isozyme). Each bar represents mean value of four observations.

FIG. 6. Correlation between CYP 3A4 Content and in vitro methemoglobin formation by NPC1161B with different individual lots of human liver microsomes. X axis values—CYP 3A4 Content (Testosterone 6β-hydroxylase activity, pmol/min/mg protein). Y axis values—Methemoglobin formation (%).

FIG. 7. Effect of erythromycin on generation of methemoglobenemic metabolites from 8-aminoquinolines in presence of pooled human liver microsomes. Concentration of ketoconazole tested were 10, 30 and 100 μM. Concentration of 8-aminoquinolines was 100 μM. Each bar shows % methemoglobin formation and values are mean of four observations.

FIG. 8. Effect of troleandomycin on generation of methemoglobenemic metabolites from 8-aminoquinolines in presence of pooled human liver microsomes. Concentration of troleandomycin tested were 1,5 and 10 μM. Concentration of 8-aminoquinolines was 100 μM. Each bar shows % methemoglobin formation and values are mean of four observations. FIG. 9. Effect of ketoconazole on generation of methemoglobenemic metabolites from 8-aminoquinolines in presence of pooled human liver microsomes. Concentration of ketoconazole tested were 5 and 10 μM. Concentration of 8-aminoquinolines was 100 μM. Each bar shows % methemoglobin formation and values are mean of four observations.

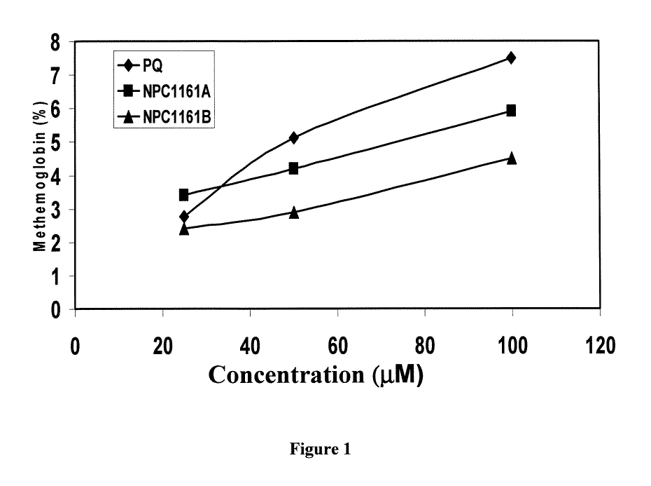

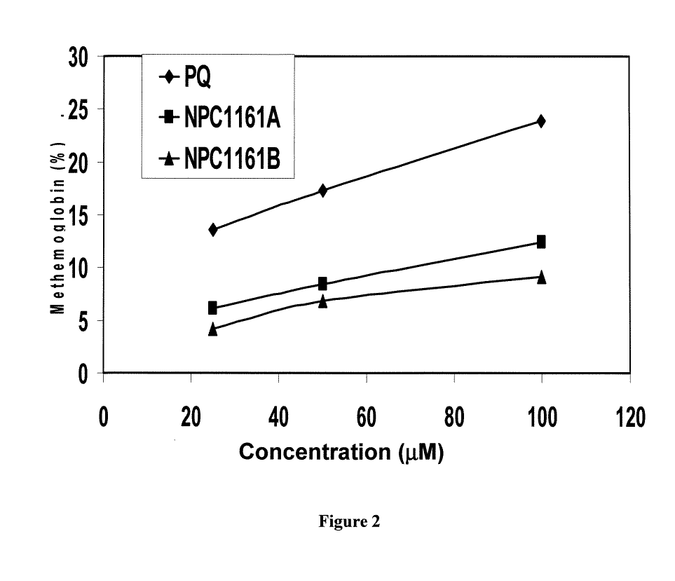

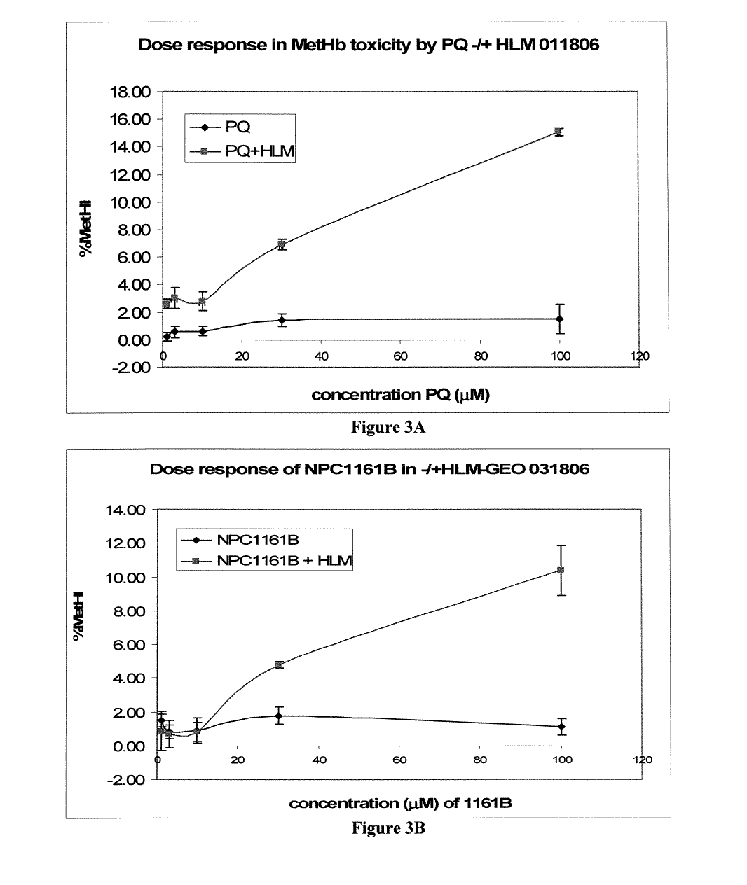

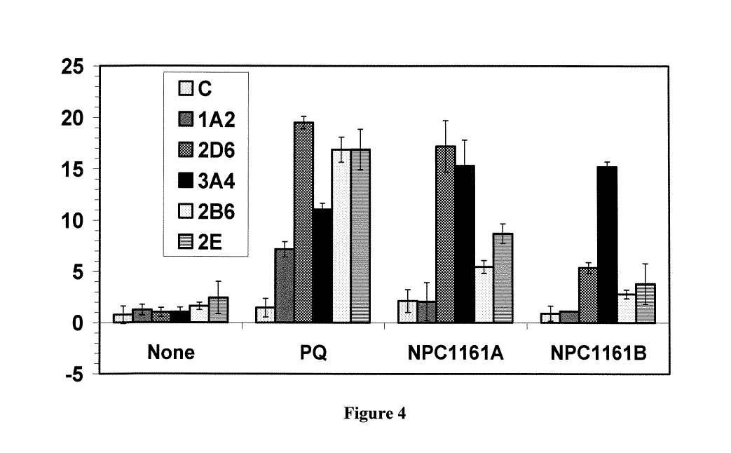

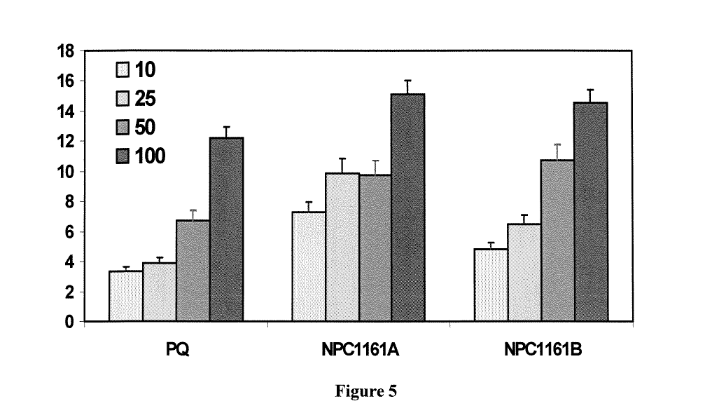

### EXAMPLES

The following example shows one embodiment of the present invention. As such it should be considered as exemplary of the present invention and not be considered to be limiting thereof.

Methemoglobin Toxicity of NPC1161B Mediated by Different Lots of Human Liver Microsomes with Variable CYP 3A4 Contents: Correlation Analysis

It was seen that variation in 3A4 content in individual microsomes leads to differential recognition of NPC1161 B by enzymes and use of 3A4 inhibitor reduces the toxicity of NPC1161B to almost negligible level. For Correlation analysis six different lots of individual human liver microsomes (HLM) were obtained from BD biosciences (www.gentest.com ) with good variations in CYP3A4 activity (Testosterone 6β-hydroxylase activity ranging from 700-12,000 pmol/mg protein). Total CYP450 content in all the six HLM lots were almost comparable and ranged between 180-570 pmol/mg protein. Donor information, genotypes, immunoquantitation was provided along with the characterization table, with different cytochrome P450 assays conducted with NADPH regenerating system, MgCl₂ and 0.1M potassium phosphate buffer.

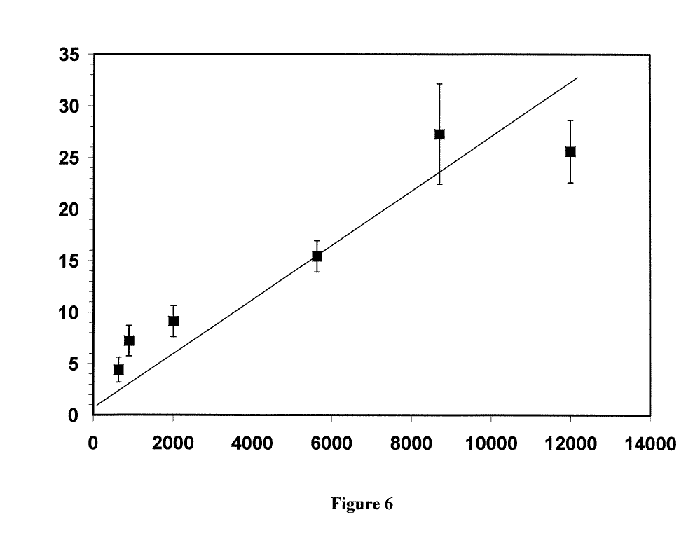

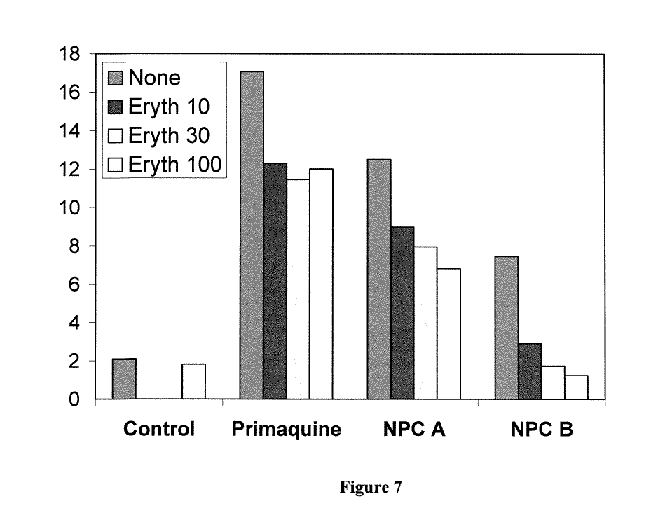

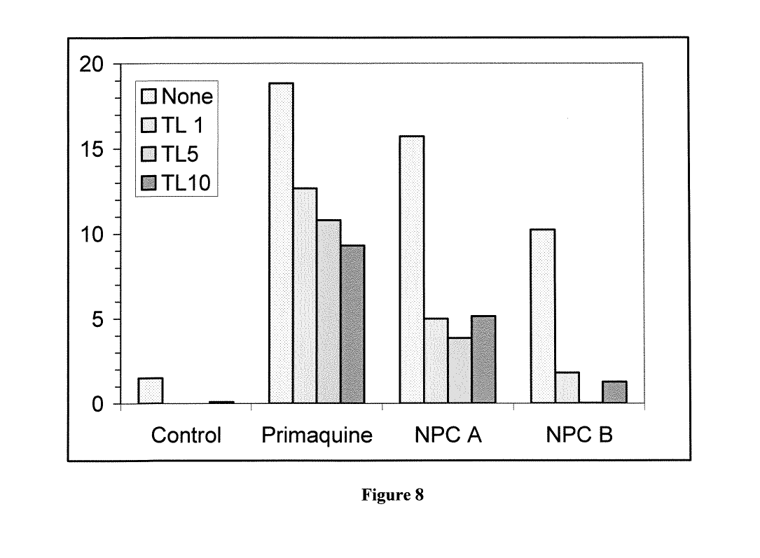

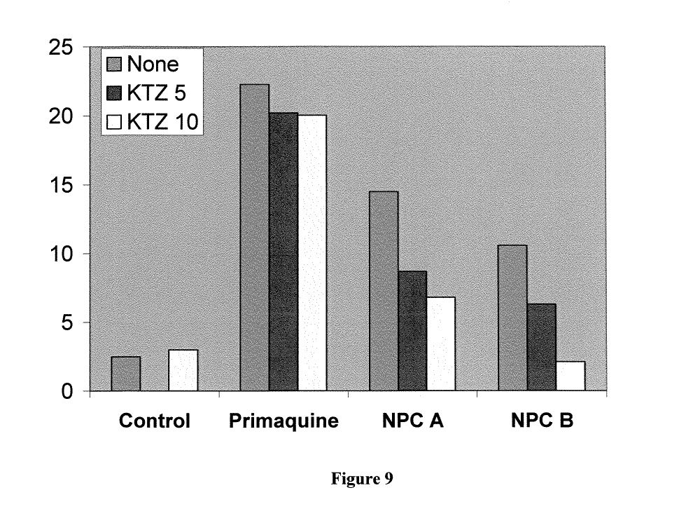

In accordance to our hypothesis CYP3A4 is the predominant enzyme metabolizing NPC1161B leading to the methemoglobin formation and use of 3A4 inhibitor reduces the toxicity to negligible amount. This is proved by a linear increase in methemoglobin toxicity with increase in 3A4 content (FIG. 6). Microsomes alone and troleandomycin control did not cause significant methemoglobin formation (Table 3). Negligible formation of methemoglobin was observed by preincubation of 10 μM of troleandomycin with all the lots of human liver microsomes. The lot of HLM (lot #452095) with least activity of CYP3A4 generated lowest levels of methemoglobin with NPC1161B while the HLM preparations with highest activity of CYP 3A4 (lot # 452018 and 452171) resulted in the highest methemoglobin toxicity of NPC1161B (Table 3).

TABLE 1
In vitro evaluation of formation of methemoglobinemic
metabolite from 8-aminoquinolines

| | | Pooled Human liver | Mouse Liver |
|---|---|---|---|
| Drug² | Control¹ | Microsomes¹ | Microsomes¹ |
| None | 1.035 ± 0.174 | 2.039 ± 0.419 | 2.715 ± 0.956 |
| Primaquine | 1.465 ± 0.414 | 17.060 ± 1.015 | 36.660 ± 2.612 |
| NPC 1161A | 2.138 ± 0.624 | 12.510 ± 1.890 | 17.790 ± 1.460 |
| NPC1161 B | 0.980 ± 0.439 | 7.455 ± 0.879 | 11.240 ± 3.612 |
| Hydroxylamine | 46.570 ± 2.194 | — | — |

¹Values are given as % methemoglobin formation and are presented as Mean ± S.D. of four observations.
²Concentration of the drug/compound was 100 μM.
Microsomes equivalent to 25 pmoles of cytochrome P450 were used in each assay.

TABLE 2
In vitro evaluation of formation of methemoglobinemic metabolite from 8-
aminoquinolines with Baculosomes ® with different human CYP isoenzymes

| Supersomes | None | Primaquine | NPC1161A | NPC1161B |
|---|---|---|---|---|
| None | 0.768 ± 0.414 | 1.465 ± 0.183 | 2.138 ± 0.624 | 0.980 ± 0.493 |
| CYP 1A2 | 1.250 ± 0.540 | 7.170 ± 0.744 | 2.060 ± 0.185 | 0.00 |
| CYP 2D6 | 1.020 ± 0.009 | 19.500 ± 1.728 | 17.200 ± 1.088 | 5.380 ± 1.402 |
| CYP 3A4 | 1.042 ± 0.146 | 11.050 ± 1.754 | 15.320 ± 0.504 | 15.280 ± 1.077 |
| CPY 2B6 | 1.625 ± 0.359 | 16.880 ± 1.215 | 5.465 ± 0.625 | 2.855 ± 0.419 |
| CYP 2E1 | 2.448 ± 1.785 | 16.890 ± 1.978 | 8.703 ± 0.956 | 3.801 ± 1.996 |
| CYP 2A6 | 2.424 ± 1.133 | 2.435 ± 0.786 | 2.775 ± 0.175 | 2.135 ± 0.530 |
| CYP 2C8 | 0.818 ± 0.203 | 2.025 ± 0.222 | 1.893 ± 0.275 | 1.353 ± 0.791 |
| CYP 2C19 | 1.235 ± 0.789 | 2.300 ± 0.816 | 1.733 ± 0.327 | 3.870 ± 0.577 |
| CYP 2C9 | 0.880 ± 0.450 | 0.450 ± 0.530 | 0.070 ± 0.130 | 3.690 ± 1.660 |

Values given are % methemoglobin formed and are mean ± S.D. of four observations.
Baculosomes ® are microsomes prepared from insect cells infected with recombinant baculovirus containing a cDNA insert for human a specific CYP isozyme and NADPH-P450 reductase.

TABLE 3
In vitro methemoglobin formation by NPC1161B with different lots of Human
Liver microsomes: Correlation with CYP3A4 content.

| | Human Liver Microsomes (Lot No) | | | | | |
|---|---|---|---|---|---|---|
| Parameter | 452095 | 452013 | 452093 | 452164 | 452174 | 452018 |

CYP Profile¹

| | | | | | | |
|---|---|---|---|---|---|---|
| Total P450 | 230/270 | 180 | 330/310 | 570 | 480/430/370 | 370 |
| (Omura & Sato) | | | | | | |
| pmol P450/mg | | | | | | |
| of total protein | | | | | | |
| CYP 3A4 | 760/630 | 890 | 2000/1500 | 5600 | 8700/8900/ | 12000 |
| (Testosterone | | | | | 8500 | |
| 6β-hydroxylase) | | | | | | |
| CYP 2D6 | 160/130 | 120 | 48/41 | 120 | 34/27/25 | 28 |
| (Bufuralol 1′- | | | | | | |
| hydroxylase) | | | | | | |
| CYP 2E1 | 1200/1300 | 2000 | 1900/2100 | 2300 | 2800/2900/ | 1200 |
| (Chlorzoxazone | | | | | 2600 | |
| 6-hydroxylase) | | | | | | |

In vitro Methemoglobin Formation (%) (Mean ± S.D.)²

| | | | | | | |
|---|---|---|---|---|---|---|
| Only | 3.34 ± 2.25 | 1.17 ± 1.43 | 1.41 ± 1.06 | 2.10 ± 1.43 | 1.37 ± 1.72 | 1.14 ± 1.20 |
| Microsomes | | | | | | |
| Microsomes + Troleandomycin | 3.18 ± 1.31 | 1.98 ± 0.98 | 3.23 ± 2.47 | 4.12 ± 0.98 | 3.31 ± 0.79 | 2.90 ± 0.75 |
| Microsomes + NPC1161B | 4.41 ± 1.21 | 7.77 ± 1.49 | 9.12 ± 1.51 | 15.44 ± 1.52 | 27.30 ± 4.85 | 25.63 ± 3.02 |
| Microsomes + NPC1161B + | 2.69 ± 1.68 | 3.90 ± 1.45 | 6.37 ± 1.03 | 7.84 ± 1.49 | 6.10 ± 1.94 | 4.68 ± 1.80 |
| Troleandomycin | | | | | | |

¹CYP profiles values for individual lots of human liver microsomes are provided by BD Bioscience (www.gentest.com).
²MtHb formation values are Mean ± S.D. of two separate experiments with four values in each experiment. Concentration of NPC1161B was 100 μM and troleandomycin

Correlation analysis between % methemoglobin formation and CYP3A4 Content of HLM preparation yielded a linear correlation with R value of >0.9 (FIG. 6). Methemoglobin toxicity of NPC1161 B did not correlate with either CYP 2D6 or CYP2E1 contents of HLM preparations. The HLM lot #452092, which contained highest activity of CYP 2D6 resulted in the lowest methemoglobin toxicity of NPC1161B. All the HLM lots did not show much variation in CYP 2E1 activity and still showed marked variation in methemoglobin toxicity of NPC 1161B.

TABLE 4
Effect of macrolide antibiotics on formation of methemoglobenemic
metabolites from 8-aminoquinoline in presence of Mouse Liver Microsomes.

| | Control | Primaquine | NPC1161A | NPC1161B |
|---|---|---|---|---|
| None | 1.198 ± 0.295 | — | — | — |
| Microsomes | 2.715 ± 0.956 | 36.660 ± 2.612 | 17.790 ± 1.460 | 11.24 ± 3.612 |
| Azithromycin | 6.598 ± 0.505 | 11.800 ± 2.862 | 10.850 ± 2.134 | 9.993 ± 1.306 |
| Erythromycin | 6.230 ± 1.009 | 23.770 ± 5.826 | 9.375 ± 0.686 | 3.133 ± 0.963 |

TABLE 5
Effect of macrolide antibiotics on formation of methemoglobenemic
metabolites from 8-aminoquinoline in presence
of pooled human Liver Microsomes.

| | Control | Primaquine | NPC1161A | NPC1161B |
|---|---|---|---|---|
| None | 0.722 ± 0.490 | — | — | — |
| Microsomes | 0.660 ± 0.591 | 17.490 ± 0.913 | 12.860 ± 1.994 | 7.697 ± 1.392 |
| Azithromycin | 0 | 12.930 ± 2.515 | 10.595 ± 0.842 | 7.565 ± 1.276 |
| Erythromycin | 0 | 13.688 ± 0.470 | 6.720 ± 1.011 | 0 |

TABLE 6
Effect of inhibitors of CYP 3A4 on generation of methemoglobenemic
metabolites from 8-aminoquinolines in presence of 3A4 Baculosomes ®

| Drug | Control | Primaquine | NPC1161A | NPC1161B |
|---|---|---|---|---|
| None | 1.985 ± 0.727 | 17.865 ± 0.8911 | 20.925 ± 0.502 | 18.185 ± 2.809 |
| Ketoconazole | — | 20.505 ± 1.260 | 19.010 ± 0.870 | 9.615 ± 1.552 |
| Cimetidine | 1.012 ± 0.845 | 21.590 ± 1.351 | 11.905 ± 1.420 | 8.567 ± 0.600 |
| Erythromycin | — | 19.243 ± 3.539 | 15.288 ± 2.359 | 11.655 ± 1.818 |
| Troleandomycin | — | 21.748 ± 0.913 | 16.743 ± 1.401 | 2.785 ± 0.628 |

Values given are % methemoglobin formed and are mean ± S.D. of four observations.
Baculosomes ® are microsomes prepared from insect cells infected with recombinant baculovirus containing a cDNA insert for human a specific CYP isozyme and NADPH-P450 reductase.
Concentration of 8-aminoquinoline - 100 μM; Concentration of inhibitor- 100 μM

One concern with this method of controlling 8-aminoquinoline toxicity is that the inhibition metabolism of the drug may impair its antiparasitic efficacy. As shown in Table 7, troleandomycin, the clinically used mechanism-based inhibitor of CYP 3A4, does not antagonize the antimalarial property of NPC1161B as assessed in mice infected with Plasmodium berghei, the malaria parasite. Thus treatment of the individuals with NPC1161B along with an inhibitor of CYP3A4 should not compromise the therapeutic efficiency of NPC1161B.

TABLE 7
In vivo antimalarial evaluation of NPC1161B in combination
with Troleandomycin in Plasmodium berghei-mouse malaria model

| | Day | | |
|---|---|---|---|
| Group | p.i | (%) Parasitemia | |
| Control | 7 | 22.2/3.9/16.7/16.9/9.8 (13.9) | |
| | 10 | 39.5/12/28.5/27.5/11.82 (23.86) | |
| | 14 | 42.4/26.8/50.8/11.7/39.6 (34.2) | |
| | 21 | 52.1 | |
| | 28 | | |
| | deaths | 3/14; 1/19; 1/21 (MST days —16.4) | |
| Vehicle Control | 7 | 26.2/9.09/24.5/16.8/(19.1) | |
| | 10 | 40.1//13.5//26.1 (26.5) | |
| | 14 | 46/33.1/50.4 (43.1) | |
| | 21 | 39.5/52.3 | |
| | 28 | | |
| | deaths | 1/7; 1/9; 1/15; 2/22 (MST days 15) | |
| Troleandomycine (TAO) i.p. | 7 | 13.3/18.3/25.5/16.5/13.1 (17.3) | |
| (50 mg/kg × 3) once daily | 10 | 22.7/34.5/32.0/32.1/24.3 (29.1) | |
| | 14 | 31.5/41.5/28.5/49.3/49.3 (40.0) | |
| | 21 | | |
| | 28 | | |
| | deaths | 5/15 (MST days 15) | |
| NPC 1161B mg/kg (Oral) | Alone | | +TAO (i.p.) |
| 2.5 × 3 | 7 | 0/0/0/0/0 | 0/0/0/0/0 |
| | 10 | 0/0/0/0/0 | 0/0/0/0/0 |
| | 14 | 0/0/0/0/0 | 0/0/0/0/0 |
| | 21 | 0/0/0/0/0 | 0/0/0/0/0 |
| | 28 | 0/0/0/0/0 | 0/0/0/0/0 |
| | deaths | nil | nil |
| 1.25 × 3 | 7 | 0/0/0/0/0 | 0/0/0/0/0 |
| | 10 | 0/0/0/0/0 | 0/0/0/0/0 |
| | 14 | 0/0/0/0/0 | 0/0/0/0/0 |
| | 21 | 0/0/0/0/0 | 0/0/0/0/0 |
| | 28 | 0/0/0/0/0 | 0/0/0/0/0 |
| | deaths | nil | nil |
| 0.625 × 3 | 7 | 0/0/0/0/0 | 0/0/0/+/0 |
| | 10 | 0/0/0/0/+ | 0/0/0/0/0 |
| | 14 | 0/+/0/0/0 | +/0/0/0.13/0 |
| | 21 | 0/+/5.2/2.6/3.7 (2.3) | 8.4/0/10.4/18.4/1.5 (7.7) |
| | 28 | 0/0/6.1/+/31.4 | 37.7/0/18.23/65.4/32.9 |
| | deaths | nil | nil |
| 0.312 × 3 | 7 | +/+/+/0/+ | +/+/0/+/0 |
| | 10 | 0/0.46/0/0/+ | 0/0/0/0/0 |
| | 14 | 0/5.8/0.66/0.8/1.36 (2.9) | 2.83/2.04/+/5.29/0 (2.0) |
| | 21 | 11.3/53.3/19.4/6.0/17.8 (21.5) | 23.8/32.4/0/28.3/0 (16.9) |
| | 28 | 44.8/74.1/74.1/60.9/53.6 | 56.3/59.4/0/49.1/0 |
| | deaths | nil | nil |

Treatment started on day 3 post infection once daily for three days

Relevant References Cited
Linares G E, Ravaschino E L, Rodriguez J B (2006). Progresses in the field of drug design to combat tropical protozoan parasitic diseases. Current Medicinal Chemistry.13:335-360.
Tekwani B L and Walker L A (2006) 8-Aminoquinolines: Future role as Antiprotozoals, Current Opinion in Infectious Diseases, 19(6):623-631.
Coleman M D, Coleman N A (1996), Drug-induced methaemoglobinaemia. Treatment issues. Drug Safety, 14:394-405
Brueckner R P, Ohrt C, Baird J K et al. (2001) 8-Aminoquinolines. In-Antimalarial chemotherapy, mechanism of action, resistance and new directions in drug discovery. (Ed Rosenthal P J), Humana Press New Jersey. pp 123-151.
Zhou S, Yung Chan S, Cher Goh B, Chan E, Duan W, Huang M, McLeod H L. (2005) Mechanism-based inhibition of cytochrome P450 3A4 by therapeutic drugs. Clin Pharmacokinet.44(3):279-304
Zhou S, Chan E, Lim L Y, Boelsterli U A, Li S C, Wang J, Zhang Q, Huang M, Xu A. (2004) Therapeutic drugs that behave as mechanism-based inhibitors of cytochrome P450 3A4. Curr Drug Metab. 5(5):415-42.

The invention thus being described, it would be obvious that the same may be varied in many ways without departing from the scope of the present invention. All such variations as would be obvious to one of ordinary skill in the art are considered as being part of the present invention.

All publications cited herein are hereby incorporated by reference in their entirety.

1. A method of controlling toxicity to a user caused by administration to the user of an aminoquinoline compound comprising administering to the user a toxicity controlling amount of at least one inhibitor of a cytochrome P450 (CYP) enzyme.

2. Method according to claim 1, wherein the CYP enzyme is at least one of CYP3A4, CYP1A2, CYP2D6, CYP2B6 or CYP2E1.

3. Method according to claim 2, wherein the inhibitor is a CYP3A4 inhibitor.

4. Method according to claim 3, wherein the inhibitor is at at least one of macrolide antibiotics, cimetidine, ketoconazole, HIV-protease inhibitor, fluoroquinolone antibacterial agents, and naturally occuring inhibitors including Schisandra fruit [Schisandra chinensis Baillon], grapefruit, and their components.

5. Method according to any one of claims 1-4 wherein the aminoquinoline compound is an 8-aminoquinoline.

6. Method according to claim 5 wherein the 8-aminoquinoline is primaquine or its analogs, or one of the enantiomers of 8-[(4-Amino-1-methylbutyl)amino]-5-(3,4-dichlorophenoxy)-6-methoxy-4-methylquinoline (NPC1161 A or B) or the racemic form of 8-[(4-Amino-1-methylbutyl)amino]-5-(3,4-dichlorophenoxy)-6-methoxy-4-methylquinoline.

7. Method of claim 1, wherein the toxicity controlled is methemoglobin toxicity or hemolysis or other hematotoxicity or other toxicities mediated by CYP-mediated oxidative metabolism.

8. Method of claim 1, wherein the toxicity controlling agent and the aminoquinoline are co-administered.

9. Method of claim 1, wherein the inhibitor and aminoquinoline are administered separately.

10. Method of claim 1, wherein at least one inhibitor is administered in an amount effective for inhibition of CYP-mediated drug metabolism.

11. Method of claim 1 wherein the at least one inhibitor and at least one aminoquinoline are administered orally.

12. A composition comprising at least one aminoquinoline and at least one cytochrome P450 (CYP) enzyme inhibitor and acceptable carriers or excipients.

13. A two package composition which is adapted for administration together comprising a first composition comprising at least one 8-aminoquinoline and an acceptable carrier or excipient and a second composition comprising at least one cytochrome P450 (CYP) enzyme inhibitor and an acceptable carrier or excipient.

14. The composition of any one of claims 12 or 13 wherein the aminoquinoline is an 8-aminoquinoline.

15. The composition according to claim 12 or claim 13 or claim 14 wherein the inhibitor is at least one of macrolide antibiotics, cimetidine, ketoconazole, fluoroquinolone antibacterials, or a HIV-protease inhibitor.

16. The composition of any one of claims 12 or 13 wherein the inhibitor is an inhibitor of at least one of CYP3A4, CYP1A2, CYP2D6, CYP2B6 or CYP2E1.

17. The composition of claim 16, wherein the inhibitor is a CYP3A4 inhibitor.
